(19) United States
(12) Patent Application Publication
(10) Pub. No.: US 2008/0161932 A1
(43) Pub. Date: Jul. 3, 2008

(54) Artificial Disc

(76) Inventors: Kevin Armstrong, Irving, CA (US); Michael Tooke, Greensboro, NC (US)

Correspondence Address: VON SIMSON & CHIN, 62 WILLIAM STREET, 6TH FLOOR, NEW YORK, NY, 10005, US

(21) Appl. No.: 11/965,515

(22) Filed: Dec. 27, 2007

Related U.S. Application Data
(60) Provisional application No. 60/882,063, filed on Dec. 27, 2006.

(51) Int. Cl. A61F 2/44

(52) U.S. Cl. 623/17.16; 623/17.14

(57) ABSTRACT

An artificial disc implant is described that comprises of two plates that have articulating surfaces interfacing with each other. In between the plates and surrounding the articulating surface is a flexible and elastic insert puck. The insert puck will have a hole that allows the two articulating surfaces to interact with one another. The articulating surfaces will also be shaped to allow different ranges of angular movements between the two endplates depending on the direction of the motion. The insert puck will provide protection to the articulating surfaces at the point of interaction and will provide cushion to the plates as they move relative to one another.

### CROSS REFERENCES TO RELATED APPLICATIONS

This application claims priority to provisional application No. 60/882,063 filed on Dec. 27, 2006, the entire disclosure of which is hereby incorporated by reference.

### TECHNICAL FIELD

The present invention relates to artificial disc implants.

### BACKGROUND OF THE INVENTION

Artificial disc technologies can be categorized into two distinct concepts: (1) articulating and (2) compressive discs. Articulating discs are quite similar to total joint replacements (hips, knees, etc.) in that they both use similar bony attachment and articulating surface technologies. An articulating disc is made up of 2 or more components which freely slide or articulate relative to one another in an attempt to mimic the motion segment. These components consist of a top and bottom endplate having some type of porous surface for attachment to the bony endplate of the vertebra usually through some sort of porous coated surface allowing for tissue ingrowth. Almost all of the top and bottom endplate designs are either cobalt chrome or titanium. Depending on the articulating surface of choice, typically either polyethylene, metal or ceramic, there may or may not be an insert in between those endplates. Any disc that uses an articulating surface/material different from the endplate, by design, must have an insert. For instance, poly on metal, ceramic on metal, ceramic on ceramic-all must have an insert. Those that use a metal on metal surface have the option of not using an insert.

In addition, the articulating surfaces of these types of discs are either constrained or unconstrained. Constraint refers to the degree to which the implant articulation can move in the different bending planes (flexion/extension, lateral bending, and axial rotation) and sheer (sliding relative to each other in anterior/posterior and/or side/side). A constrained implant will be most restrictive to motion in one or more of these planes while an unconstrained implant will have few limitations.

In contrast, compression discs have two metal plates for attachment to the vertebral endplates, but instead of an articulating surface, there is a spongy, elastic material sandwiched in between the plates. This spongy material is attached to the endplate using some adhesive or process to meld the two components together. There are a variety of materials which can be used for this material.

Currently discs are implanted through the same anterior approach and have surgical risks. Implanting a three component disc is surgically easier than putting in a one or two piece design. This is because the third piece allows the surgeon to manipulate the endplates rather than the bone in placing the third piece within the spinal column. There can be advantages to putting in discs through approaches other than an anterior approach.

In spinal implants, there are several problems that can result in failure of the implant: (a) dislocation of the bearing surface and (b) expulsion of the implant itself and (c) destruction of the bearing surface. These problems can occur during the normal movement of the human and is often due to improper placement of the disc within the spinal column. Due to the improper placement, forces are exerted on the disc that it was not expected to handle and consequently the articulating surfaces become misaligned or the entire disc or part of it is forced out of the disc space.

Another problem with articulating discs relates to auto fusing, which is a phenomenon that occurs when soft tissue or other bodily material grows into the disc in a manner that effectively fuses the joint. This renders the implant useless and is a potential occurrence in any articulating implant with an open articulation.

Another issue with articulating discs is a matter of support and normal resistance to bending moments. A normal disc has a compressive material with an inherent resistance to bending. When an artificial disc is implanted, the diseased disc is removed along with the supporting structure. When the articulating disc is implanted, there is no constraint to motion around the center point. This puts more strain on adjacent motion segments, musculature and ligaments and has an undetermined effect on the future performance of the implant. A final issue is that a hard bearing surfaced implant does not allow compression which is allowed in a healthy disc.

Yet another problem that exists with articulating discs is wear debris. During normal human movement with an articulating disc implant, the two endplates will constantly rub against one another. This friction can cause tiny fragments and/or pieces to come off either endplate. Depending on the material used, these fragments can cause problems in the body.

Compressive discs suffer a number of disadvantages as well. The idea behind choosing a compressive disc lies in an effort to make the disc as similar as possible to a healthy disc in terms of the allowed motion: both compressive as well as bending. A major problem of CDs is their ability to last the cycles a disc is expected to endure. During normal human activity, discs will move relative to one another in all directions. At the interface between the elastomer compressive material and the hard bearing plate, the connective material between the two will have to withstand all of the forces due to these motions. In particular, sheer forces will tear the elastomer compressive material from the disc at the point where the material is attached to the disc or the elastomer will delaminate. Sheer forces are forces that move the elastomer compressive material is a sideways direction compared to the endplate.

### SUMMARY OF THE INVENTION

The present invention solves the problems of the prior art by creating an articulating disc implant encased in an elastomeric material. One embodiment of the invention comprises two plates that have articulating surfaces interfacing with each other. In addition, in the spaces between the two plates, there is a insert puck. The insert puck will surround and encapsulate the entire point of contact between the articulating surfaces. The insert puck will generally have elastic and compressive properties. The articulating surfaces can have different configurations, such as a dual radius of differing lengths. This variation can provide for a more normal articulation in both flexion/extension and lateral bending and a high degree of constraint. This invention gives the following benefits due to the following characteristics:
1) Anterior/posterior motion is limited thereby reducing forces on the posterior elements and reducing the sheer forces on the elastomeric material due to the articulation of the two plates.
2) An articulation that more closely mimics articulation in both the anterior/posterior and lateral bending planes due to the dual radius design.
3) A resistance to bending moments which more closely mimics the resistive and/or supportive forces of a healthy disc due to the inclusion of the elastomer material.
4) An articulation that is encapsulated by a protective elastomer disc will serve the following purposes:
   a. Retain any wear debris from the bearing surfaces to within the encapsulation
   b. Prevent or eliminate autofusing of the articulation.
   c. Prevent dislocation of the articulating surfaces.
   d. Prevent the elastomeric disc from being expunged from the disc.

### BRIEF DESCRIPTION OF DRAWINGS

FIG. 1a is a perspective view of one embodiment of the present invention fully assembled.

FIG. 1b is a perspective view of the three components separated from one another in accordance with one embodiment of the present invention.

FIG. 2a is a top view of the three components in accordance with one embodiment of the present invention.

FIG. 2b is a front cutout view along line A of the three components in accordance with one embodiment of the present invention.

FIG. 2c is a side cutout view along line B of the three components in accordance with one embodiment of the present invention.

FIG. 3a is a bottom view of the top plate in accordance with one embodiment of the present invention.

FIG. 3b is a top view of the top plate in accordance with one embodiment of the present invention.

FIG. 3c is a front view of the top plate in accordance with one embodiment of the present invention.

FIG. 3d is a side view of the top plate in accordance with one embodiment with the present invention.

FIG. 3e is a front view of the top plate in accordance with another embodiment of the present invention.

FIG. 4a is a bottom view of the bottom plate in accordance with one embodiment of the present invention.

FIG. 4b is a top view of the bottom plate in accordance with one embodiment of the present invention.

FIG. 4c is a front view of the bottom plate in accordance with one embodiment of the present invention.

FIG. 4d is a side view of the bottom plate in accordance with one embodiment of the present invention.

FIG. 4e is a front view of the bottom plate in accordance with another embodiment of the present invention.

FIG. 5a is a top view of the insert puck in accordance with one embodiment of the present invention.

FIG. 5b is a front view of the insert puck in accordance with one embodiment of the present invention.

FIG. 5c is a side view of the insert puck in accordance with one embodiment of the present invention.

FIG. 6a is a perspective view of a five component device in accordance with another embodiment of the present invention

FIG. 6b is a perspective view of the five components separated from one another in accordance with another embodiment of the present invention.

FIG. 7a is a top view of the five components in accordance with another embodiment of the present invention.

FIG. 7b is a front cutout view along line A of the five components in accordance with another embodiment of the present invention.

FIG. 7c is a side cutout view along line B of the five components in accordance with another embodiment of the present invention.

FIG. 7d is a side cutout view along line A of the five components in accordance with another embodiment of the present invention.

FIG. 8a is a side cutout view of the artificial disc in accordance with another embodiment of the present invention.

FIG. 8b is a side cutout view of the artificial disc in accordance with another embodiment of the present invention.

### DETAILED DESCRIPTION OF THE DRAWINGS

For the purposes of understanding the invention, reference will now be made to the embodiments illustrated in the drawings. It will be understood that no limitation of the scope of the invention is thereby intended. Any alterations and further modifications in the described embodiments, and any further applications of the principles of the invention as described herein are contemplated as would normally occur to one skilled in the art to which the invention relates.

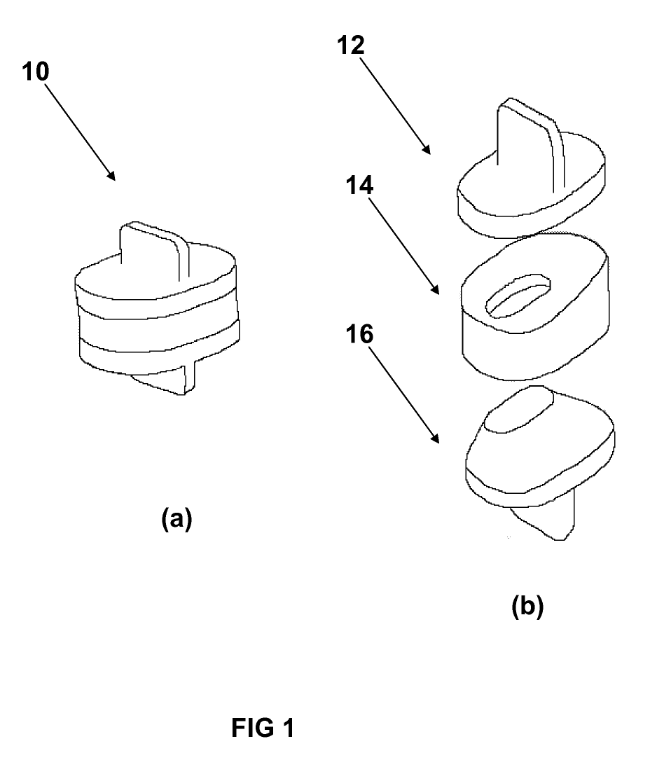

Referring to FIGS. 1a and 1b, one embodiment of the invented disc 10 is depicted. The invented disc 10 is composed of three components, a top plate 12, an insert puck 14 and a bottom plate 16. As can be seen in FIG. 1a, the three components are designed to be able to fit together in a linear direction. Insert puck 14 has a hole in its interior portion such that the bottom surface of top plate 12 and the top surface of bottom plate 16 will interact with each other.

The top and bottom plates can be composed of any hard durable material such as cobalt chrome, titanium, ceramics, polymers, plastics or any combinations of these. There are some advantages and disadvantages to using different materials. For example, in some instances titanium is favored because it is friendlier to the human body. However, since titanium is a softer material it will also be more subject to wear and create more wear debris than other materials. The insert puck can be composed of any softer material that has suitable compressive and expansive properties.

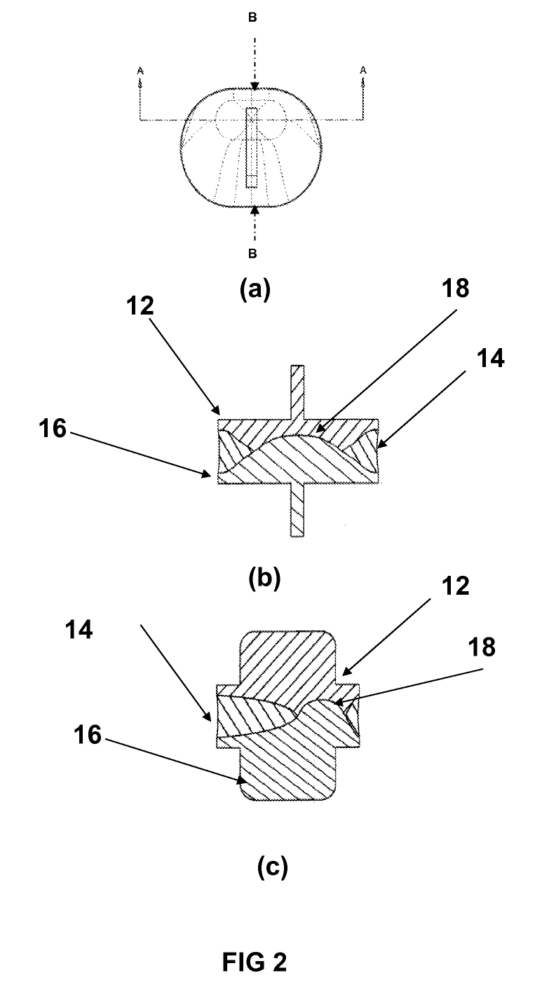

FIG. 2a shows a top view of the invented disc 10 with the edges of the three components outlined within the body of the device. The silhouette of the device can be an oval shape that corresponds to the normal size of the disc. Other shapes of the device can be used depending on the nature of the implant. FIG. 2b shows a front cutout view along arrow A and FIG. 2c shows a side cutout view along arrow B.

As can be seen in FIG. 2b, the top plate 12 and bottom plate 16 interface together at an articulation point 18. The interface between the top and bottom plate operate in a ball and socket relationship, which allows the top plate to swivel around the bottom plate. Other types of endplate interfaces can be used as long as movement is allowed between the plates.

As can be seen in FIG. 2b, the top endplate can swivel relative to bottom endplate in a side to side direction. The range of motion in this swivel direction will be constrained by two things: (1) the radius of the articulating surface of the bottom piece and (2) the insert puck 14 that surrounds the interface.

The radius of the articulating surface of the bottom piece will limit the range of motion that the top piece can move. If the top plate swivels too far to the side, then the edges of the end plate will contact each other to prevent further motion. As shown in FIG. 2b, the articulating surface of the bottom endplate has a rather relatively large radius. A larger radius greatly constrains the range of angular movement of the two endplates before the edges touch. This approximates the normal range of human motion since the body is less flexible in bending sideways.

In addition, the position of the point of contact 18 of the articulating surfaces of the top and bottom plates are located in the center of the disc. This also approximates normal human movement as there is neither favored motion to either the left or right side. It will be appreciated that the length of the radius and the positioning of the articulating surfaces can be changed to facilitate the need to approximate expected movements of the human user. This protection will also affect the choice of materials allowed as softer materials such as titanium can be used without the dangers of wear debris or autofusion.

The insert puck 14 surrounds the point of contact 18 and will also constrain the range of swiveling motions that the top and bottom plates 12 and 16 will be able to accomplish. Thus, in addition to the length of the radius and the positioning of the point of contact, the selection of the compressive material also allows the ability to control the range of motion. A less elastic and compressive material will provide greater resistance to the angular movement in that direction.

FIG. 2c shows a cutout side view along arrow B from FIG. 2a. As can be seen in FIG. 2c, the top plate 12 and bottom plate 16 also interface together at one point in a ball and socket relationship in the front and back direction. As such, the top endplate can swivel relative to the bottom endplate in a front to back direction. Similar to the side to side motion, the range of motion in this swivel direction will be constrained by two things: (1) the radius of the articulating surface of the top piece and (2) the insert puck 14 that surrounds the interface.

FIG. 2c depicts a rather relatively small radius because the front to back swiveling movement of the disc is expected to be greater in normal human movement. In addition, the position of the articulating surfaces of the top and bottom plates are located further toward the posterior. This positioning also approximates normal human movement as forward movement is favored over backward movement. Having a different radius and position of the point of contact 18 from the sideways direction also helps to control rotation of the top endplate around the ball of the bottom endplate. It will be appreciated that the radius and positioning of the articulating surfaces can be changed to facilitate the need to approximate expected movements of the human user.

In addition, while FIGS. 2b and 2c shows that the entire side, front and back areas are filled with the insert puck 14, it is possible to partially fill the area to control range of movement. The partial filling could be in either or all directions, but having the insert puck 14 completely surround and encapsulate the point of contact 18 creates a physical barrier to the articulation interface. That barrier effect will help to retain any wear debris from the bearing surfaces and prevent autofusing of the articulation. In addition, it will help prevent dislocation of the articulating surfaces.

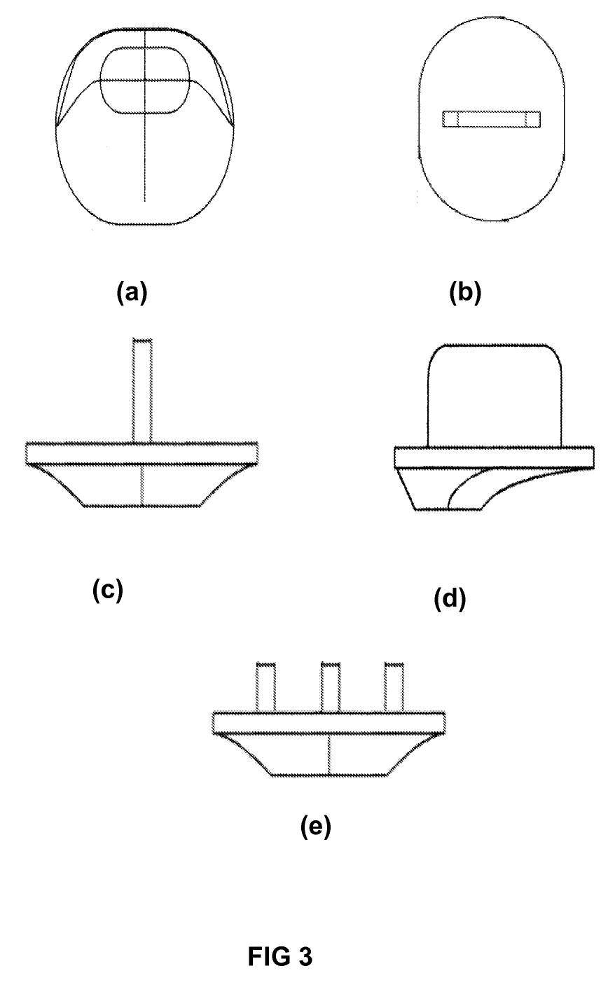

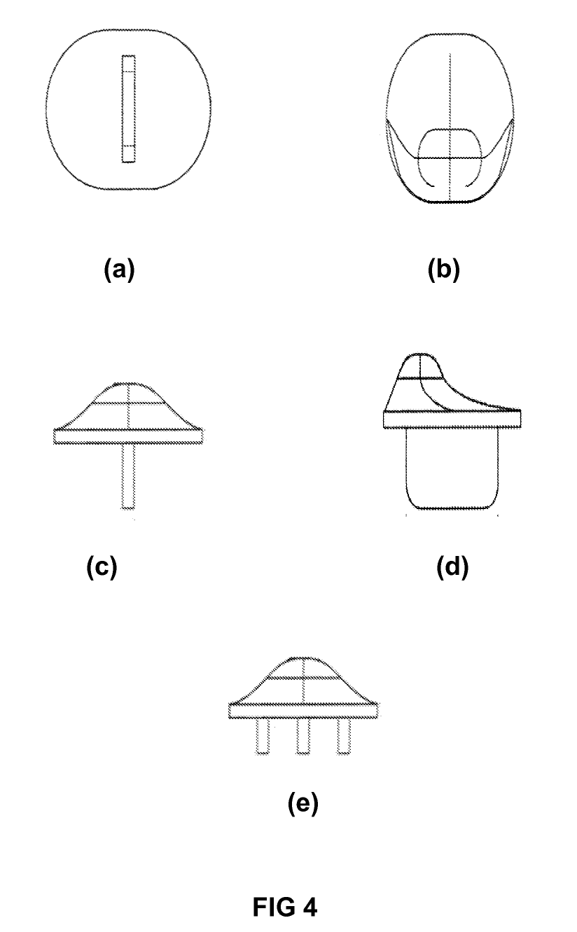

FIGS. 3a-d depicts the top, bottom, front and side views of the top plate 16. FIGS. 4a-d depicts the top, bottom, front and side views of the top plate 16. As can be seen from these figures, the top and bottom endplates have a keel on the top and bottom sides respectively. These keels will be inserted into channels cut into the vertebral body. The height of the keels can be changed to fit the circumstances. A shown in FIGS. 3(e) and 4(e), an alternative design will have multiple keels that are shorter in height. Such a design will help in the implantation process by allowing the surgeon the ability to adjust to the spatial difference in patients. In addition, shorter keels will be less damaging to the patient as it does not require deep cuts into the attaching bone. By having several points of contact with the bone, it will also create a more stable disc by distributing any torque forces impacting the disc and human bone.

Referring back to FIGS. 3(a)-(d) and 4(a)-(d), it can be seen that both interior surfaces have sloping surfaces to accommodate the insert puck 14. The sloping surfaces will facilitate smoother operation of the disc and prevent any needless wear and tear that can occur with sharp corners. It should be noted that the top endplate has the socket as the articulating surface. This allows the top endplate to move while it swivels around the ball of the bottom endplate. This also approximates the normal human movement.

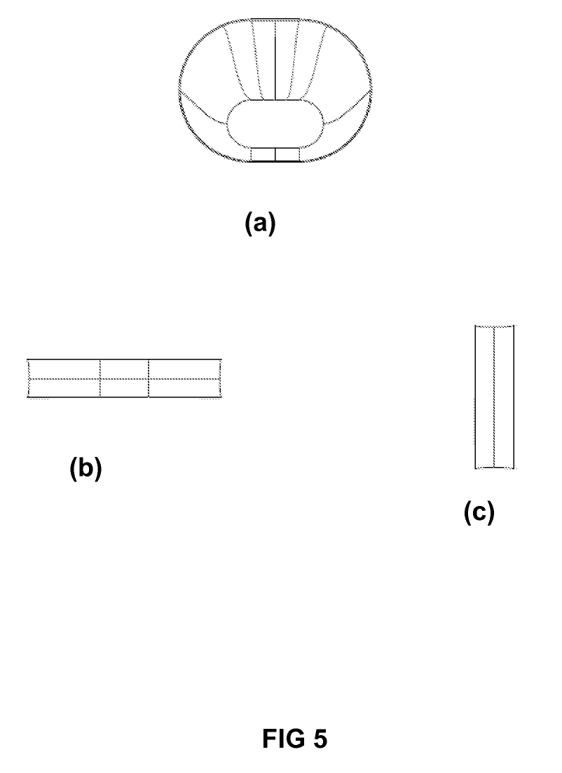

FIGS. 5(a)-(c) depict the top, front and side view of the insert puck 14. The insert puck has a hole through the interior that allows the articulating surfaces of the top and bottom endplates to contact one another. By having a hole through the insert puck 14, the insert puck 14 will be held in place by the articulating surfaces to prevent expulsion of the insert puck 14.

Alternatively, insert puck 14 can merely have an indentation instead of a hole. The indentation will be in both the top and bottom of the insert puck to accommodate the top and bottom surfaces of the bottom. The indentation should be deep enough to allow the top and bottom plates to have a lever point to support articulating movement as between the two plates.

The insert puck 14 is the same size as the top and bottom endplates. However, the insert puck and be made smaller to allow greater movement between the endplates. In addition, the insert puck 14 can be composed of different materials that have different compression/elastic properties to also impact the range of movement of the endplates. For example a more compressive and elastic material can be placed in the area corresponding to the front of the insert puck to allow the user a greater ability to bend forward. The insert puck is shown as an integral unit, but it can be assembled from more than one piece.

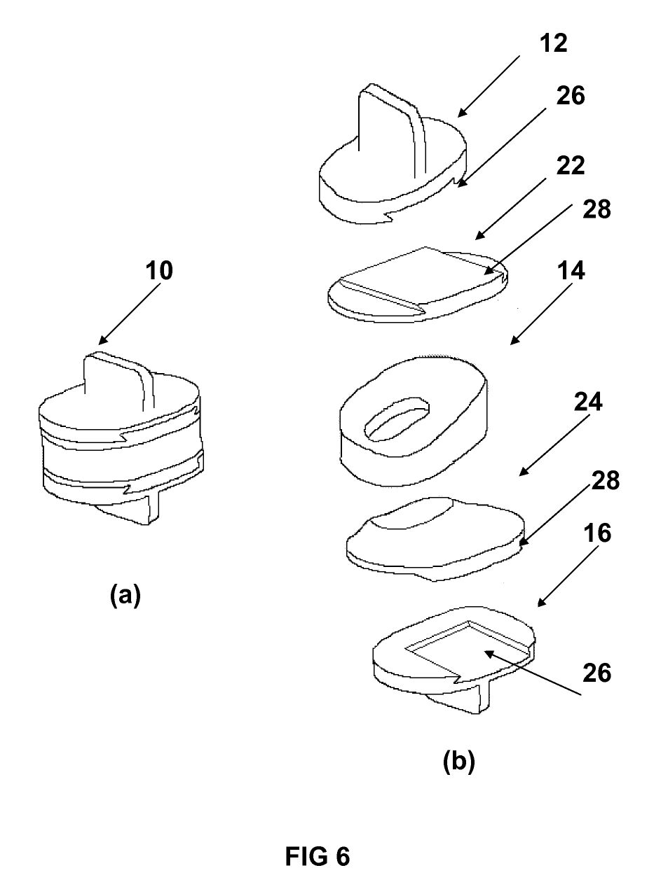

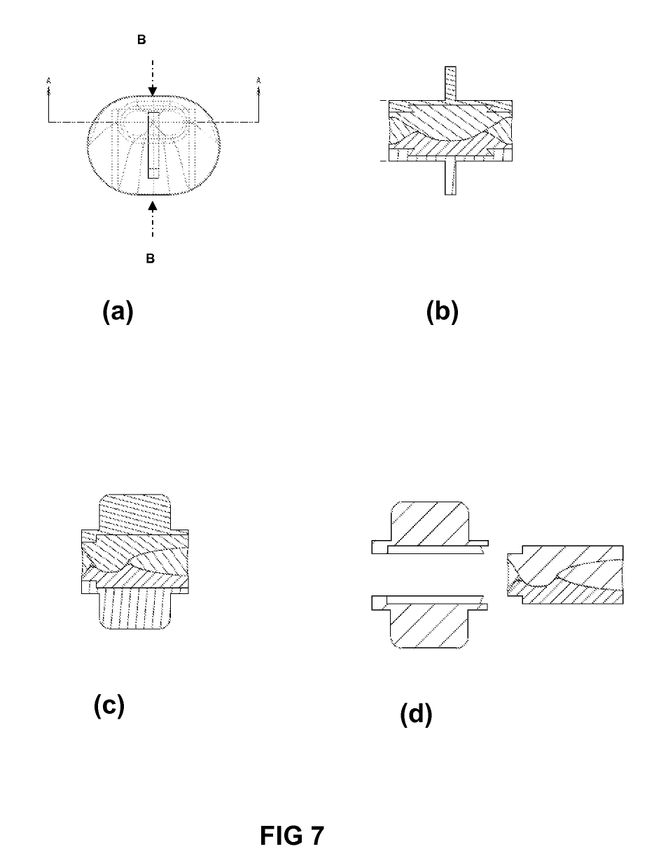

FIGS. 6(a) and (b) show an alternate embodiment to the three component artificial disc. Similar to FIG. 1(a), FIG. 6a shows the device fully assembled. However, the device depicted in FIG. 6(a) shows a five piece assembly. The insert puck 14 is the same as the three piece assembly, but bearing portions 22, 24 will be attached to the endplates 12, 16. The respective endplates 12, 16 will have beveled indentations 26 that will receive the bearing portions. The bearing portions 22, 24 will have corresponding protrusions 28 that will fit into the beveled indentations 26. This creates a modular design.

Having a modular approach has two distinct advantages. First, the implantation process will be made easier. The surgeon will be able to first implant the endplates into the bone. A distractor would spread the endplates and the bearing portions 22, 24 could be slid into the respective endplates 12, 16. The second advantage is that it allows the surgeon the ability to choose different bearing portions to fit the situation during the operation. Bearing portions made of different heights and/or different materials can be selected and used after the endplates have been implanted into the bone.

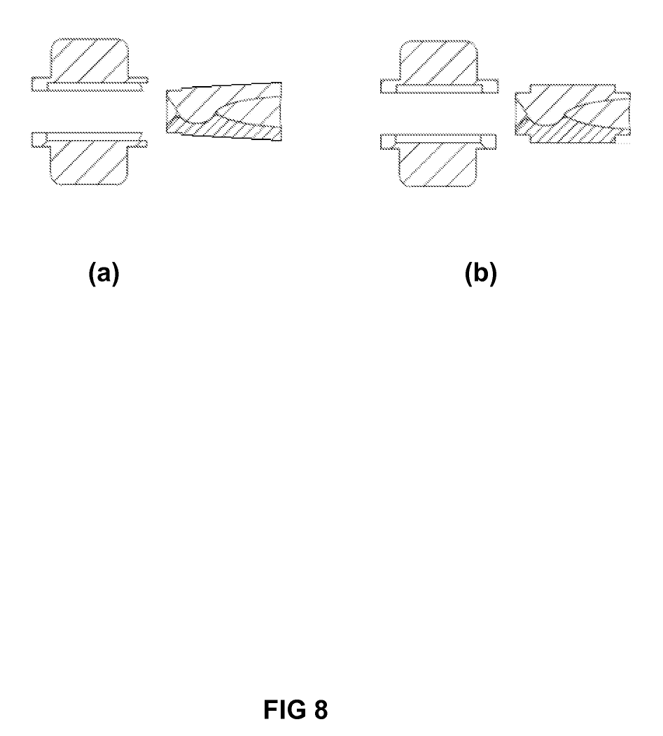

As shown in FIG. 8(a), an alternative embodiment to this five piece modular approach could include having the indentation on the endplates and the protrusions on the bearing portions have a slope. With the slope, the bearing portions will act as a wedge to allow easier insertion into the space between the endplates. FIG. 8(b) shows yet another embodiment where the bearing portions 22, 24 will not be slid into the endplates 12, 16. Instead the bearing portions will have protrusions that are designed to be inserted into a cavities in the endplates 12, 16. The surgeon will simply “pop” the bearing portions into place.

Through the positioning of the point of articulation, the relative lengths of radii in the side and front/back direction and the characteristics of the compressive insert, the invented disc will solve many of the disadvantages of the prior art. The dual radius design of the point of articulation allows better mimicry of normal human use and provides less strain on the muscles and ligaments. Having both the point of articulation and the compressive insert duplicates the natural bending and resistance effect that exists in a normal healthy disc while prolonging the life of the artificial disc 10 by reducing the sheer effect with the point of articulation.

We claim:

1. An artificial disc implant comprising:
(1) a top endplate having a first articulating surface;
(2) a bottom endplate having a second articulating surface that is capable of interfacing with said first articulating surface when said top endplate is placed on top of said bottom endplate; wherein said endplates are capable of angular movement through said first and second articulating surfaces;
(3) an insert puck that is capable of surrounding the first and second articulating surfaces when said first and second articulating surfaces are interfacing with one another;
(4) wherein said articulating surfaces allow for a different range of angular movement in a first direction than a second direction; said first direction being approximately perpendicular to said second direction

2. The artificial disc implant recited in claim 1, wherein said articulating surfaces interface in a ball and socket relationship.

3. The artificial disc implant as recited in claim 2, wherein said articulating surface of said top piece operates as the socket and said articulating surface operates as the ball.

4. The artificial disc implant as recited in claim 3, wherein said articulating surface of said bottom piece has a different radius in said first direction than said second direction.

5. The artificial disc implant as recited in claim 1, wherein said insert puck has elastic and compressive characteristics.

6. The artificial disc implant as recited in claim 1, wherein said top and bottom endplates have an anchoring keel; said anchoring keel being located on the opposite side of said articulating surfaces.

7. The artificial disc implant as recited in claim 6, wherein said articulating surfaces of said top and bottom endplates are capable of being detached and attached to said top and bottom endplates.

8. The artificial disc implant as recited in claim 1 wherein said articulating surfaces will interface at a point corresponding to the center of said endplates in said first direction and not in the center of said endplates in said second direction.

9. The artificial disc implant as recited in claim 1 wherein said top and bottom endplates are comprised of cobalt chromium.

10. The artificial disc implant as recited in claim 1 wherein said insert puck comprises of two different material with different elastic and compressive characteristics.

11. The artificial disc implant as recited in claim 1 wherein said first and second articulating surfaces contact each other through a hole in said insert puck.

12. An artificial disc implant having a left, right, posterior and anterior sides, said disc implant comprising:
(a) a first plate having a top, bottom, left, right, posterior and anterior sides; said first plate having an articulating surface on said bottom side;
(b) a second plate having a top, bottom, left, right, posterior and anterior sides; said second plate having an articulating surface on said top side; said articulating surface on said second plate being capable of interacting with said articulating surface on said first plate to allow movement when said first plate is aligned with said second plate such that said posterior side of said first plate is aligned with said posterior side of said second plate and said anterior side of said first plate is aligned with said anterior side of said second plate;
(c) an insert puck capable of being placed in between said first and second plates when said first and second plates are aligned such that said articulating surfaces of said first and second plates are covered by said insert puck at the point of interaction.

13. The artificial disc implant as recited in claim 12 wherein said movement is angular movement in a posterior to anterior direction and angular movement in a left to right direction.

14. The artificial disc implant as recited in claim 13 wherein the range of angular movement in said posterior to anterior direction is different from the range of angular movement in said left to right direction.

15. The artificial disc implant as recited in claim 14 wherein said different ranges of angular movement is caused by the shape of the articulating surfaces.

16. The artificial disc implant as recited in claim 12 wherein said articulating surfaces operate in a ball and socket relationship.

17. The artificial disc implant as recited in claim 12 wherein said articulating surfaces interact in a location closer to said posterior side than said anterior side.

18. The artificial disc implant as recited in claim 12 further comprising an implant keel on top of said first plate and an implant keel on bottom of said second plate.

19. The artificial disc implant as recited in claim 18 wherein said first plate is comprised of an implant endplate portion and a bearing portion; said bearing portion being detachable from said endplate portion; said articulating surface being located on said bearing portion and said implant keel being located on said endplate portion.

20. An artificial disc implant comprising:
a first endplate having an articulating surface;
a second endplate having an articulating surface capable of interfacing with said articulating surface of said first endplate when said first endplate is place on top of said second endplate; the interface between said articulating surfaces allowing angular movement between the endplates in at least two directions and wherein the range of angular movement in one direction is different from the range of angular movement in the other direction.

21. The artificial disc implant as recited in claim 20, wherein said interface between said articulating surfaces operate in a ball and socket relationship.

22. The artificial disc implant as recited in claim 21 wherein the radius of said ball is different in one direction than the radius in the other direction.

23. The artificial disc implant as recited in claim 20 further comprising an insert puck that is capable of surrounding the interface between said articulating surfaces.

24. The artificial disc implant as recited in claim 23 wherein said insert puck is comprised of a unitary body and said articulating surfaces contact each other through a hole in said insert puck when interfacing with each other.
